(19) United States
(12) Patent Application Publication
(10) Pub. No.: US 2025/0302347 A1
(43) Pub. Date: Oct. 2, 2025

(54) SYSTEMS AND METHODS FOR ONE-POINT, ON-SITE CALIBRATION OF SENSORS

(71) Applicant: NERv Technology Inc., Kitchener (CA)

(72) Inventors: Ricky Tjandra, Kitchener (CA); Abdallah Hassen El-Falou, Kitchener (CA); Kenan Habib, Kitchener (CA); Khaled Berry, Kitchener (CA); Lauren Janine LeSergent, Waterloo (CA)

(21) Appl. No.: 19/094,163

(22) Filed: Mar. 28, 2025

Related U.S. Application Data
(60) Provisional application No. 63/571,028, filed on Mar. 28, 2024.

(51) Int. Cl. A61B 5/1495; A61B 5/145

(52) U.S. Cl. CPC A61B 5/1495; A61B 5/14546

(57) ABSTRACT

Disclosed herein are systems and methods for a single-point, on-site calibration of one or more sensors in an inline sensor device, the method comprising: calibrating, at a first location, each of the one or more sensors with a plurality of variable concentration reference calibrators to generate multi-point calibration data; calibrating, at a second location, each of the one or more sensors using a single-concentration reference calibrator to generate single-point calibration data; translating, by at least one processor communicatively coupled to a memory, the multi-point calibration data and the single-point calibration data to translated calibration data; and employing, at the second location, the translated calibration data to analyze one or more analytes detected by one or more calibrated sensors in the sensor device.

### CROSS REFERENCE TO RELATED APPLICATIONS

This application claims priority from U.S. Provisional Patent Application No. 63/571,028, filed on Mar. 28, 2024, the disclosure of which is hereby incorporated by reference.

### FIELD OF THE DISCLOSURE

The present disclosure relates generally to the field of the calibration of sensors, specifically to a system and method for in-situ calibration, more specifically to a method for in-situ calibration of biosensors.

### BACKGROUND

Sensors are often used to monitor important biomarkers in patients' physiological fluids to assess their health status. It is advantageous to have a sensor that can measure the biomarkers in-situ, without having to draw samples or disconnect the sensor from the patient, due to a variety of reasons such as infection control. It is due to this need that several technologies have emerged to enable in-situ, bedside monitoring of a vast number of patient biomarkers.

One of the challenges for in-situ biomarker monitoring sensors is the need for biosensors to be calibrated periodically in order to maintain their accuracy. The typical calibration process involves removal of the sensor from the patient, exposure to a known reference material and re-attachment to the patient again.

Typically, calibrating a sensor comprises making measurements with the sensor of calibration samples of known analyte concentration or blanks (samples comprising only the matrix, and no analyte), so that the measured signal strength of the sensor may be correlated with the known analyte concentrations, in order to provide a calibration curve, wherein measured signals of samples having unknown analyte concentrations' signals may be plotted on the calibration curve, in order to interpolate the analyte concentration.

Calibration of sensors is important to get the most accurate and reliable readings of analytes present in a patient's biofluids, especially for medical devices. Practically, there is a trade-off between resources and accuracy that needs to be considered when choosing how to calibrate sensors.

For point-of-care medical devices, it is desirable to use calibration that is the least burdensome possible due to the limited resources of healthcare providers. Calibrations that are simple to perform will increase compliance with calibration methods and reduce human-induced errors.

In terms of accuracy, the gold standard for calibration of sensors is a multi-point calibration. For example, a 3-point calibration has at least 3 points of calibration: end range concentrations (one low and one high) and a mid-point concentration, between the end range concentrations. The more calibration points, the more accurate the calibration will be. Multi-point calibrations, while accurate, can be burdensome on the end-user. This burden only increases when multiple sensors are present on the same device.

In the prior art, there may exist some bioanalyte sensors having more streamlined methods of calibration.

There exist some examples in the prior art of devices that “self-calibrate”; however, they generally are not equipped to receive continuous flows of fluid from a patient, and are not capable of being calibrated for more than one analyte (i.e., only glucose, or only pH). Blood gas analyzers (BGAs), for example, uses onboard cartridges to perform-self calibration. This requires a lot of complex systems that is not suitable for the inline device form factor.

Point of Care (POC) blood gas analyzers (EPOC) may use removable cartridges. These sensors have a smaller form factor but still require fluid pumps, and other internal system tools to operate. They can be mobile, but not small enough to be used inline with patients.

Generally, BGAs also measure relatively narrow ranges of analytes which translate into smaller numbers of calibrators. These methods are not conducive to measuring wider concentration ranges.

There remains a need, therefore, for calibration methods for on-site, inline sensors, which are able to provide accurate assessments of analyte concentration, while maintaining simplicity for the end-user during calibration.

Any discussion of the related art throughout the specification should in no way be considered as an admission that such related art is widely known or forms part of common general knowledge in the field.

### BRIEF SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which is explicitly or implicitly described by the following description and claims.

It is an object of the disclosure to provide a system and method for systems and methods for one-point, on-site calibration of sensors.

In accordance with an aspect of the disclosure, there is provided a method for calibrating one or more sensors in a sensor device fluidically in-line with a patient, the method comprising: calibrating, at a first location, each of the one or more sensors with a plurality of variable concentration reference calibrators to generate multi-point calibration data; calibrating, at a second location, each of the one or more sensors using a single-concentration reference calibrator to generate single-point calibration data; translating, by at least one processor communicatively coupled to a memory, the multi-point calibration data and the single-point calibration data to translated calibration data; and employing, at the second location, the translated calibration data to analyze one or more analytes detected by one or more calibrated sensors in the sensor device.

In some embodiments, each calibrating step comprises validating calibration accuracy of the one or more sensors.

In some embodiments, the translating step further comprises performing one or more of: slope corrections, offset corrections, temperature corrections, and drift corrections.

In some embodiments, the method further comprises determining, prior to calibrating each of the one or more sensors, types of the one or more sensors, the plurality of variable concentration reference calibrators, and the one or more single concentration reference calibrators.

In some embodiments, the method further comprises determining, prior to calibrating each of the one or more sensors, a sequence in which each of the reference calibrators from the plurality of variable concentration reference calibrators are applied to the one or more sensors, wherein the sequence is based on cross sensitivities of the one or more sensors with each of the reference calibrators from the plurality of variable concentration reference calibrators.

In some embodiments, the method further comprises determining, prior to calibrating each of the one or more sensors, a type of flow in which the plurality of variable concentration reference calibrators is applied to the one or more sensors, wherein the type of flow is a continuous flow or an intermittent flow.

In some embodiments, the plurality of variable concentration reference calibrators is periodically tested to obtain current calibration values, wherein the current calibration values are retrieved prior to generating the multi-point calibration data.

In accordance with another embodiment of the disclosure, there is provided a computer-implemented method for calibrating one or more sensors in a sensor device fluidically in-line with a patient, the method comprising: storing, in a memory coupled to at least one processor, multi-point calibration data generated from calibrating, at a first location, each of the one or more sensors with a plurality of variable concentration reference calibrators; storing, in the memory coupled to the at least one processor, single-point calibration data generated from calibrating, at a second location, each of the one or more sensors using a single-concentration reference calibrator; determining, by the at least one processor, translated calibration data based on the multi-point calibration data and the single-point calibration data; and analyzing, by the at least one processor, one or more analytes detected by one or more calibrated sensors in the sensor device based on the translated calibration data.

In some embodiments, each calibrating step comprises validating calibration accuracy of the one or more sensors.

In some embodiments, the determining translated calibration data further comprises performing one or more of: slope corrections, offset corrections, temperature corrections, and drift corrections.

In some embodiments, the computer-implemented method further comprises receiving the multi-point calibration data and the single-point calibration data from a data source, wherein the data source is one of: a server through a network, the sensor device or a barcode.

In some embodiments, the computer-implemented method further comprises receiving, from the data source, pre-calibration information generated from pre-calibration steps comprising: determining types of the one or more sensors, the plurality of variable concentration reference calibrators, and the one or more single concentration reference calibrators; determining a sequence in which each of the reference calibrators from the plurality of variable concentration reference calibrators are applied to the one or more sensors; and determining a type of flow in which the plurality of variable concentration reference calibrators is applied to the one or more sensors.

In accordance with another embodiment of the disclosure, there is provided a system for calibrating one or more sensors, the system comprising: a sensor device fluidically in-line with a patient comprising the one or more sensors for detecting one or more analytes; a memory; and at least one processor coupled to the memory comprising program instructions, wherein the program instructions are executable by the at least one processor to perform operations comprising: storing multi-point calibration data generated from calibrating, at a first location, each of the one or more sensors with a plurality of variable concentration reference calibrators; storing single-point calibration data generated from calibrating, at a second location, each of the one or more sensors using a single-concentration reference calibrator; retrieving the multi-point calibration data and the single-point calibration data from the memory; determining translated calibration data based on the multi-point calibration data and the single-point calibration data; and analyzing the one or more analytes detected by the one or more calibrated sensors in the sensor device based on the translated calibration data.

In some embodiments, each calibrating step comprises validating calibration accuracy of the one or more sensors.

In some embodiments, the translating step further comprises performing one or more of: slope corrections, offset corrections, temperature corrections, and drift corrections.

In some embodiments, the system further comprises a data source, wherein the data source is one of: a server through a network, the sensor device or a barcode.

In some embodiments, the operations further comprise determining, prior to calibrating each of the one or more sensors, types of the one or more sensors, the plurality of variable concentration reference calibrators, and the one or more single concentration reference calibrators.

In some embodiments, the operations further comprise determining, prior to calibrating each of the one or more sensors, a sequence in which each of the reference calibrators from the plurality of variable concentration reference calibrators are applied to the one or more sensors, wherein the sequence is based on cross sensitivities of the one or more sensors with each of the reference calibrators from the plurality of variable concentration reference calibrators.

In some embodiments, the operations further comprise determining, prior to calibrating each of the one or more sensors, a type of flow in which the plurality of variable concentration reference calibrators is applied to the one or more sensors, wherein the type of flow is a continuous flow or an intermittent flow.

In some embodiments, the plurality of variable concentration reference calibrators is periodically tested to obtain current calibration values, wherein the current calibration values are retrieved prior to generating the multi-point calibration data.

The advantages and features of the present disclosure will become better understood with reference to the following more detailed description and claims taken in conjunction with the accompanying drawings in which like elements are identified with like symbols.

### BRIEF DESCRIPTION OF THE DRAWINGS

To easily identify the discussion of any particular element or act, the most significant digit or digits in a reference number refer to the figure number in which that element is first introduced.

FIG. 1 illustrates a multi-port stopcock flow cell device in accordance with one embodiment.

FIG. 2A illustrates a flow chart of a data validation portion of a calibration method in accordance with one embodiment.

FIG. 2B illustrates a flow chart of a data calibration portion of a calibration method in accordance with one embodiment.

FIG. 2C illustrates a flow chart of a calibration artifact filtration portion of a calibration method in accordance with one embodiment.

FIG. 2D illustrates a block diagram of a method for a single point, on site calibration of a sensor device, in accordance with one embodiment.

FIG. 3 illustrates a schematic diagram of a system used to determine the calibration of a sensor device in accordance with one embodiment.

FIG. 4 illustrates a routine 400 for calibrating one or more sensors in accordance with one embodiment.

FIG. 5 illustrates a routine 500 for calibrating one or more sensors in accordance with one embodiment.

FIG. 6A illustrates a system including an inline sensor device in accordance with one embodiment.

FIG. 6B illustrates a magnified view of internal components within an inline sensor device in accordance with one embodiment.

Elements in the several drawings are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood elements that are useful or necessary in commercially feasible embodiments are often not depicted in order to facilitate a less obstructed view of these various embodiments of the present disclosure.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

When introducing elements of aspects of the disclosure or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The term “exemplary” is intended to mean “an example of.” The phrase “one or more of the following: A, B, and C” means “at least one of A and/or at least one of B and/or at least one of C.”

Systems, methods and devices disclosed herein can be utilized for monitoring, detecting and predicting different forms of postoperative complications, such as leakage, that can arise following surgeries. Embodiments can include a sensing and diagnostic device that utilizes sensors, for example, on a catheter or an inline device (sensor device), to detect or predict, for example, the presence of luminal fluid when a leak develops.

In some embodiments, systems, methods and devices disclosed herein include sensors, such as biosensors that can be used to sense bio-signal data, placed at locations proximate to the surgical site, enabling the monitoring of biological fluids for analytes that could be indicative of a surgical leak.

In some embodiments, sensors may include electrochemical or solid-state sensors with different forms, which include but are not limited to potentiometric, voltammetric, conductometric, capacitive, amperometric or ion-sensitive field effect transistors (ISFET). In some embodiments, sensors may be piezoelectric or micro-electro-mechanical systems (MEMS). Sensors may include terminals that connect to active, counter, reference or pseudo-reference electrodes depending on the type of sensor being utilized. Sensors can be of different types that include but are not limited to pH sensors, ion-sensitive sensors, temperature sensors, lactate sensors, electrolyte sensors, impedance sensors, fluid sensors, light-based sensors, microorganism sensors, protein sensors, inflammatory sensors, carbohydrate sensors, enzyme sensors, oxygen sensors such as P02 (partial pressure of oxygen) sensors, amylase sensors, urea sensors, creatinine sensors, pressure sensors and flow sensors.

Sensors may be connected in series or in parallel, and may be disposed sequentially, for example, along a length of a fluid channel.

In some embodiments, sensors may include a temperature sensor, such as a thermistor.

In use, a thermistor may undergo changes in resistance correlated to change in temperature. Thus, a temperature may be determined by determining a resistance of the thermistor, by exciting with current and measuring voltage (or vice versa).

A temperature sensor may be used to account for a number of artifacts and error sources in the bio-signal measurements. A temperature sensor may be used to compensate or modulate signals from other sensors that are temperature dependent such as impedance and pH. A rise in fluid temperature detected by temperature sensor can indicated an influx of new fluid, as biological fluids tend to have higher temperatures relative to ambient temperatures.

An array of temperature sensors and a heating element may be used to measure fluid flow rate using the principles of thermal mass fluid transport.

In some embodiments, sensors may include a flow sensor such as a flowmeter to measure the volumetric or mass flow rate of a fluid such as a liquid or a gas, for example, in a patient's body.

In some embodiments, sensors may include a pH sensor that is electrochemical in nature allowing biological analytes to be transduced into electrical signals that can be then measured, monitored and analyzed to determine if a postoperative complication is developing. A system of interdigitated electrodes (active, counter and reference) may be fabricated on a biocompatible substrate. The electrodes may be fabricated from biocompatible materials: gold, platinum, titanium and silver, and then later functionalized with either an ion sensitive field effect transistor (ISFET) or an active polyaniline (PANI) polyaniline/polyurethane (PAIN/PU), polyurethane, polymer or other suitable layer. In an example, p-biosensors are 500 pm×500 μm in size, allowing them to be placed on catheters to monitor changes in pH over time.

A pH sensor may be formed from a conducting polymer made from Aniline monomers. A sensitivity to pH levels of a suitable conducting polymer can allow for its use as a pH sensitive component in pH sensors.

A pH sensor may be calibrated and/or controlled by a potentiostat, in particular, an electronic device that controls the difference in potential and current of a 3-electrode system comprising of a working electrode (WE), a reference electrode (RE) as well as a counter electrode (CE). This electrical instrument has many applications that may be used to fabricate a pH sensor such as Cyclic Voltammetry (CV), Chronoamperometry and Chronopotentiometry.

A pH sensor may be configured to detect a pH value within a threshold or boundaries, or deviation from such boundaries.

In some embodiments, sensors may include a light-based sensor, such as photoelectric sensors, utilizing a combination of light transmitters or sources and detectors in the ultraviolet to infrared spectrum to measure the fluid's light absorption or transmission characteristics. Single-wavelength or multi-wavelength rays may be used.

Light-based sensors can include a combination of light transmitters and detectors in the ultraviolet to infrared spectrum and be used to measure a fluid's light absorption or transmission characteristics.

Light absorption or transmission characteristics can be indicative of changes in the bodily and luminal fluids that can include, but are not limited to, protein composition and concentration, pH, conductivity, inflammatory markers and cellular activities due to onset of complications or disease. This also enables measurement of the fluid's color, which can be indicative of bleeding (red), bile leaks (green-yellow), fecal leaks (brown), gastric leaks (green), urine leaks (yellow), and other fluids of specific colors.

In some embodiments, single-wavelength or multi-wavelength rays may be used. Changes detected in the absorption or transmission characteristics of fluids within specific light bands or wavelength may enable measurement of a fluid's color. Since serous fluids (for example, peritoneal and pleural fluids) are typically pale yellow, a change in color may be indicative of bleeding (red), bile leaks (green-yellow), fecal leaks (brown), gastric leaks (green), urine leaks (yellow), or other fluids of specific colors.

In some embodiments, a light-based sensor may include a combination of light transmitters and detectors in the ultraviolet to infrared spectrum to measure the scattering of light by the fluid to measure its turbidity. Serous fluids are typically clear in appearance and low in turbidity. An increase in turbidity, for example, as measured as an increase in the light measured by a photodetector at right angle, may be indicative of white blood cells and microorganisms within the fluid, which may be due to infection.

A light-based sensor may include multiple light sources and receivers. For instance, a single broadband light source may be used in combination with multiple band-specific photodiodes (e.g. red, green and blue). In this way, the absorption/transmission characteristics of the fluid can be measured across as many bands as there are photodetectors present. Similarly, multiple light sources may be utilized in combination with a single broadband photodetector, whereby each light source is turned on successively and the transmitted light measured accordingly by the photodetector. Lastly, light sources and photodetectors may also utilize dynamic filters to allow the emission or detection of specific bands of light in lieu of multiple sources or photodetectors.

In some embodiments, sensors include an impedance sensor typically operated with an alternating current (AC) excitation which may be used to evaluate the patient status. An impedance sensor may include an electrode pair, and include an excitation and readout circuit.

In some embodiments, an impedance sensor may be configured to perform AC excitation within a well-defined and constant fluid geometry (constrained by a channel or housing), allowing a normalized impedance (or specific impedance) and admittance to be determined.

In some embodiments, a fluid's impedance may be measured across a range of frequencies (ranging from Hz to MHz) to separate the contribution of individual electrolytes and infer the ionic composition of the fluid. A user condition may be based at least in part on the fluid's ionic composition.

Measured impedance values may be transformed to determine a conductivity (for example, real element of the impedance) of a fluid. Conductivity may reveal a characteristic of the fluid itself, and hence may directly serve a clinical value. For example, conductivity may indicate an analyte's inherent characteristics and composition.

Impedance may be affected by fluid volume and geometry, and thus measured impedance may be used to localize and track particles and bubbles in a fluid channel.

In some embodiments, an impedance sensor may be used to account for a number of artifacts and error sources in bio-signal measurements.

In some embodiments, an impedance sensor may be used to detect a rapid and drastic increase in impedance beyond the range of bodily fluids which may be indicative of the presence of air bubbles in the channel. Air bubbles are a challenge to catheter-based measurements as they cause artifacts with readings.

In some embodiments, an impedance sensor may be used to detect a sudden increase in impedance, which may be indicative of a presence and a quantity of non-homogenous substances and particles (e.g., blood clots, fibrin).

In some embodiments, an impedance sensor may be used to detect blood coagulation (typically characterized by a sudden increase in impedance, followed by a slower but sustained increase in impedance), and hence, the presence of blood and risk of channel blockage.

In some embodiments, an array of impedance sensors placed along the channel may be used to detect and track air bubbles, non-homogenous substances, and/or particles as they travel through the channel, using techniques described herein.

In some embodiments, sensors may include amylase sensors.

In use, systems, methods and devices may monitor for trends and changes in physical and chemical biomarkers that may include but are not limited to pH, temperature, fluid flow, pressure, lactate, lactic acid, nitrates, glucose, alkali ions, oxygen, bicarbonate, inflammatory proteins, bacterial proteins and other biomarkers, for example, that are associated or correlated with leakage.

Single sensors or sensor arrays can be placed along the wall of a catheter, inside dedicated lumens, or in an inline device, that enable the device to detect and monitor if a leak is developing.

In some embodiments, a catheter may be used as a carrier for sensors to monitor the internal compartments of the body such as the peritoneal or pleural cavity, without applying any negative pressure. Catheter may be connected to a balloon or a mechanical pump to apply negative pressure to facilitate the drainage of fluid. A catheter may also be connected to a fluid supply such as saline solution to perform therapeutic and diagnostic functions such as dialysis or irrigation.

In some embodiments, multiple sensors may be spaced apart along a length of a catheter. Multiple sensors placed along the catheter, may allow for multiple regions to be sensed and spatial progression of a leak to be tracked.

A catheter may be formed of a tube having a hollow or solid body and made of medical grade materials, such as a suitable polymer. In some embodiments, the catheter may be a flexible substrate.

In some embodiments, a catheter may be formed of a material with low friction.

A catheter may have different designs where the catheter may be cylindrical, rectangular, flat, or T-shaped in cross-section and the catheter may have a single lumen or multiple lumens.

Sensors such as biosensors may be connected to a monitor such as an electronic data acquisition system (DAQ) that may be situated inside or outside a patient's body, which may continuously process data obtained from the sensors. The connection can be established via different methods including but not limited to, wires and connectors that may be embedded within the catheter's body or within at least one lumen designed to allow wires and connectors run through them. The connection may also be established wirelessly by transmitting the data obtained in-vivo from biosensors via a transmitting system to a receiver placed outside the body.

In some embodiments, each of multiple sensors are independently in communication with a monitor.

A monitor may have a screen allowing readouts to be directly observed on the device. A monitor may also use various visual or audio queues such as small LEDs or alarm sounds to signal various events.

Data acquired by a monitor can also be communicated to a computer system via wired or wireless media to allow further analysis and visualization. The data communicated may be processed, raw, or summarized.

In some embodiments, data collected by a monitor can be analyzed to identify trends associated with the development of different complications. This may be performed by evaluating single or multiple data sets acquired from one or more sensors over time to diagnose and determine the stage of development of the complications.

Should one or more of the sensors demonstrate biological trends that are associated with surgical leakage, an alarm signal may be sent from the monitor to a computer-based system allowing patients to determine the appropriate medical action.

In an example, a slow decrease in local pH could indicate either a small leak or poor blood supply to the wound site. If a simultaneous slow increase in lactate concentration is observed, it may indicate a lack of blood supply (i.e., ischemia). If lactate concentration is steady, it may indicate a slow leak.

In another example, a sharp decrease in pH may indicate a large leak. If the pH returns to its baseline, it may suggest that a wound is healing despite the leak. If the pH continues to drop, or remains low, it may indicate a significant leak that the body may have difficulty recovering from.

Systems and methods disclosed herein may perform monitoring, detection and diagnosis, and prediction. For example, monitoring may present data that is sensed by sensors such as biosensors. Detection and diagnosis may, by way of algorithms, detect a condition in a patient and/or make a determination of a diagnosis, such as a leak, what kind of leak it is, and where the leak came from, for example, with an associated confidence level. A prediction may use sensory data to examine different trends and process signals to predict a leak that may occur in the future, for example, with an associated confidence level. As such, embodiments of systems and methods disclosed herein may identify physiological differences between a leak occurring and precursors to a leak.

Systems and methods disclosed herein may be used to perform clinical functions. In an example, a catheter system may be connected to mechanical elements that can apply negative pressure allowing fluid to be drained from a patient's body in addition to its diagnostic function. Such clinical function can be both performed at locations in a patient's body such as inside a GI tract or in a peritoneal cavity.

Techniques for applying negative pressure may include but are not limited to balloons, mechanical pumps, vacuum systems or other devices that can suck fluid, for example, from the body to the outside. In some embodiments, fluid that is being drained may assist in diagnostic application by causing constant fluid flow across sensors. In some embodiments, a clinical function may be performed by pumping fluid into a patient's body.

The term “bodily fluid(s)” as used herein may refer to fluids originating from inside the human body, fluids that are excreted or secreted by a body (e.g., blood, gastric juice, and peritoneal fluid), and similar fluids. In extension, the term “luminal fluid” refers to a subset of bodily fluids that exist within inner cavities, intestines, vessels, tubular organs and many other membrane-bound organs such as gastric juices, intestinal fluids, fecal matter, urine, bile fluid, and other similar fluids.

The terms “biomarker(s)” and “analyte(s)” as used herein may refer to molecules, substances, and chemical or physical properties that can be measured or detected as bio-signals in bodily fluids. They include, but are not limited to, pH, temperature, electrolyte concentration, fluid flow rate, pressure, lactate, lactic acid, nitrates, alkali ions, inflammatory proteins, bacterial proteins, specific cells, molecules, genes, gene products, enzymes, hormones, inflammatory proteins, and glucose.

The terms “biosensor(s)” and “sensor(s)” as used herein may refer to a device or system that detect or react to biomarkers or bio-signals, transducing these signals into measurable electrical signals. Biosensors and sensors utilized herein may include but are not limited to pH sensors, lactate sensors, amylase sensors, lactic acid sensors, glucose sensors, temperature sensors, pressure sensors, enzymatic sensors, protein sensors, biological sensors, ion sensors, electrolyte sensors, impedance sensors, conductivity sensors, flow sensors and other forms of electrochemical and solid-state sensors.

One or more of these sensors may be employed in a sensor device which is fluidically inline with a patent (for example, by means of a fluid flow channel), such that patient biofluids flow over the one or more sensors in the sensor device and one or more analyte signals are measured. In some embodiments, the sensor device may comprise an input port attachable to a catheter for insertion in a patient's body, an output port in fluid communication with a fluid reservoir, and a fluid channel defining fluid communication between the input port and the output port, wherein the fluid channel comprises one or more sensors for continuously monitoring biomarkers or analytes identified and measured from the patient's bodily fluids. In other embodiments, the sensor device may comprise one or more interchangeable sensor modules, a processing module and a flow channel element, wherein the flow channel element defines fluid communication between an input port and output port. The flow channel element is similar to that of the aforementioned fluid channel in that it comprises one or more sensors for measuring data such as biomarkers and analytes from a patient's bodily fluids.

Disclosed herein are systems and methods for calibrating sensors or sensor devices with a one-point calibration performed on-site/at the point of care, without sacrificing the accuracy of the calibration method. The one-point calibration may also be described as a single-point calibration as described herein.

Devices and methods for carrying out the present disclosure are presented in terms of embodiments depicted within the FIGS. However, the present disclosure is not limited to the described embodiments, and a person skilled in the art will appreciate that many other embodiments of the present disclosure are possible without deviating from the basic concept of the present disclosure, and that any such work around will also fall under scope of this present disclosure. It is envisioned that other styles and configurations of the present disclosure can be easily incorporated into the teachings of the present disclosure, and the configurations shall be shown and described for purposes of clarity and disclosure and not by way of limitation of scope.

In some embodiments, the sensor device may comprise a fluid channel, the fluid channel comprising one or more sensors, by way of a multi-port stopcock flow cell device which not only allows the one or more sensors to measure biomarkers or analytes from a patient's bodily fluids, but also allows for the one or more sensors to be calibrated.

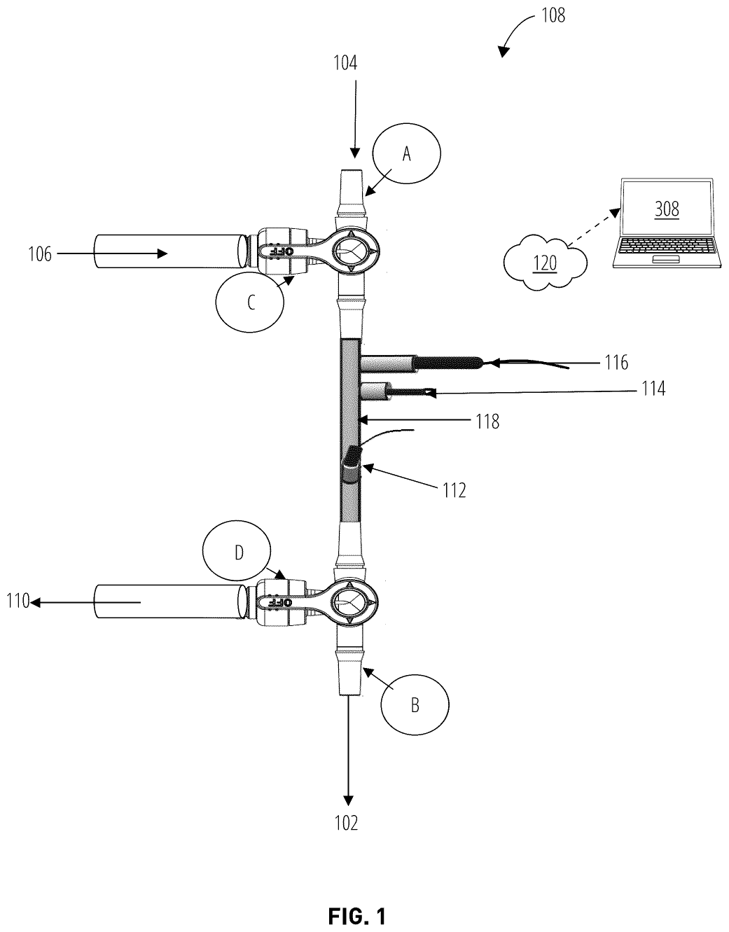

FIG. 1 illustrates a prior art multi-port stopcock flow cell device 108, according to an embodiment comprising an inlet port (A), an outlet port (B), a calibration/cleaning inlet port (C), a calibration/cleaning outlet port (D), connected by means of a flow cell 118, which comprises multiple biosensors (Biosensor e.g. pH electrode 116, biosensor e.g. pH reference electrode 114, and Biosensor e.g. Electrical conductivity (EC) 112), the biosensors capable of measuring fluid properties, biomarkers or other analytes.

Generally, the multi-port stopcock flow cell device 108 preferably comprises: one or more inlet ports (A,C), receiving fluid from a fluid source, fluidically connected by a flow cell 118 comprising one or more fluid channels, to one or more outlet ports (B,D); and a mechanism (i.e. a lever) for diverting flow of fluid between ports and fluid channels, wherein: at least one of the one or more inlet ports comprises an inlet tip, connectable to a syringe, and a slip tip, connectable to said fluid source, and at least one of the one or more outlet ports comprises an inlet tip, connectable to a syringe, and a slip tip, connectable to a reservoir.

In this embodiment, flow of the patient fluid flow in 104 and calibration/cleaning fluid flow in 106 enter from ports (A) and (C), respectively, by means of a lever, located at the junction between ports (A) and (C) as in the previously described embodiment.

Further in this embodiment, the patient fluid flow out 102 and calibration/cleaning fluid flow out 110 are controlled by an additional lever located at the junction between ports (B) and (D).

Upon fluid flowing through the flow cell 118, and over the biosensors 112, 114, 116, the biosensors measure data relating to fluid properties, and send the measured data, via a connection mechanism 120 to a computer system 312.

The connection mechanism 120 may be wired or wireless, and may include, but is not limited to, the internet, a wired connection, Bluetooth, Near Field Communication (NFC), and any other connection mechanisms known in the art. The data may be sent to cloud storage, and downloadable to the computer system 312. The computer system 312 may comprise a computer, laptop, smart phone, tablet, or any other computing device known in the art. The computer system 312 may be on-site, with the multi-port stopcock flow cell device 108, or remote from it. In an example, data may be measured at a patient's home, and automatically sent, via the internet, to a hospital or lab environment for analysis.

According to some embodiments of the disclosure, flow cells may comprise additional features such as internal vibration motors for breaking up bubbles. The multi-port stopcock flow cell device may also be in fluid communication with a patient, such as by connection to a patient catheter, for monitoring patient biofluids. Using the multi-port stopcock flow cell device allows for multiple sensor calibrations and readings, without disconnecting the device from the patient.

According to an embodiment of the disclosure, there may be provided a method for a single point, on-site calibration of a sensor device. Generally, the method comprises a first calibration step having multiple points, a second calibration step having a single point, and a translation step which corrects the first calibration step with the second calibration step (or vice versa).

Calibrating the sensor device in the factory only is typically not sufficient, as conditions vary between the point of care and the factory, the calibrator value may also drift over time, and instruments' sensor responses may vary with varying conditions, drift over time, or change for other reasons known in the art.

The single point, on-site calibration method may allow the calibration of a sensor device which is fluidically in-line with a patient, such as those disclosed by the inventors of the present disclosure, in PCT application number PCT/CA2020/050395, PCT/CA2024/050135 and U.S. patent application Ser. No. 18/456,096 (shown in FIG. 1), which are incorporated by reference herein.

Generally, the method comprises two separate calibrations—a “factory calibration” and a “point of care” calibration, and a translation step, which corrects the factory calibration with the point of care calibration.

Alternatively, the factory calibration may be used to correct the point of care calibration, with the translation step.

More specifically, the method may comprise:

1. A multi-point calibration for each of one or more sensors of the sensor device at a first location (for example, in a manufacturing facility, in a lab, at a clinic, etc.). Typically, for a given analyte, a multipoint calibration may comprise at least three calibration points (2 end range points and a mid-range point). The first location may be any location other than a point-of-care location of a patient.
2. A one-point calibration for each of the one or more sensors of the sensor device at a second location (for example, at the point of care of the patient, i.e. their home, a hospital, a clinic, etc.). In the examples given, the first location is generally separate from the second location, as the point of care may not be equipped with the calibrants or personnel necessary to perform a multi-point calibration.
3. A translation step, which translates, using a translation model, the multi-point calibration to the single-point calibration. Translation of models can be done in the form of a slope or offset correction, or a correction factor, for example. The translation step may comprise an algorithm which automatically applies the correction factor, or an algorithm which determines which correction factor is the most accurate, prior to applying the correction factor. The translation step may comprise a drift model to compensate for drift of sensors and calibrators in storage/use (e.g. tracking of lot numbers and digitally sending updated model, using machine learning, or first principles model to make a translation model, etc.). The translation model may comprise a combination of corrections, drift models, environmental-based adjustments, and the like, which are applied to convert the calibration curve from the first step, into one usable in the lab. For linear calibration curves, the amalgamation of the corrections generally results in a shift up or down of the calibration curve, although the slope may be adjusted as well.

For a robust multi-point calibration and single-point calibration, the calibration methods need to take into account the number and kinds of sensors present, the calibrators that are needed (and the interactions between them), what order or sequence to deliver them, delivery methods or type of flow which may be single continuous or intermittent (i.e. interrupted with air or some other flushing or rinsing agent), as well as the checks for the correctness of sensor data and/or calibrator values that are needed. This pre-calibration step may be automatic or manual.

Some sensor devices may comprise more than one sensor, and these may be referred to as multiplexed sensors.

If sensor devices have multiplexed sensors, order of the calibration fluids are determined based on the cross-sensitivities of the sensors with the calibrators of the other sensors. Sensors should be calibrated in ascending order of cross-sensitivities (i.e. sensors that are the most affected by the other sensors calibrators should be calibrated first, so on and so forth). For example, in a sensor device that has both electrical conductivity and pH sensors, the electrical conductivity sensors are calibrated before the pH sensors. Electrical conductivity sensors may be calibrated by exposing them to calibrators with a precise salt concentration since they are very sensitive to contamination. Conversely, pH sensors may be calibrated by calibrators that have an inherent buffering capacity and can better tolerate small amounts of contamination.

Alternatively, more than one fluid channel may be provided, such that sensors may reside in their own fluid channel to avoid contamination, and to avoid the need to calibrate in a specific order.

Between each calibrator (and prior to the first calibrator), sensors may be rinsed with alternating aliquots of air and the upcoming calibrator fluid in order to decrease the odds of contamination as much as possible.

This step of alternating aliquots of air and the calibrator fluid is especially important in sensors that are housed in milli/microfluidic channels as well as calibrators that rely on a precise concentration to maintain its nominal value (e.g. EC calibrators) as contamination is especially difficult to remove and may persist if adequate rinsing is not done.

Air may be used as a buffer in between aliquots of fluid to increase the rinsing action and remove more contamination compared to the same volume of calibrator fluid when delivered continuously (e.g. 5×1 mL aliquots of rinse fluid interspersed with 5×1 mL aliquots of air is better than 5 mL of rinse fluid followed by 5 mL of air delivered continuously). This fluid delivery process can be done manually (e.g. through using syringes, etc.) or automated by using programmable pumps.

When the rinse step has been completed and the calibrator is in contact with the sensor, there may be run a validation algorithm to determine the sensor's calibration accuracy, ensuring that the sensor has read the correct calibrator value.

These validation algorithms include but are not limited to:

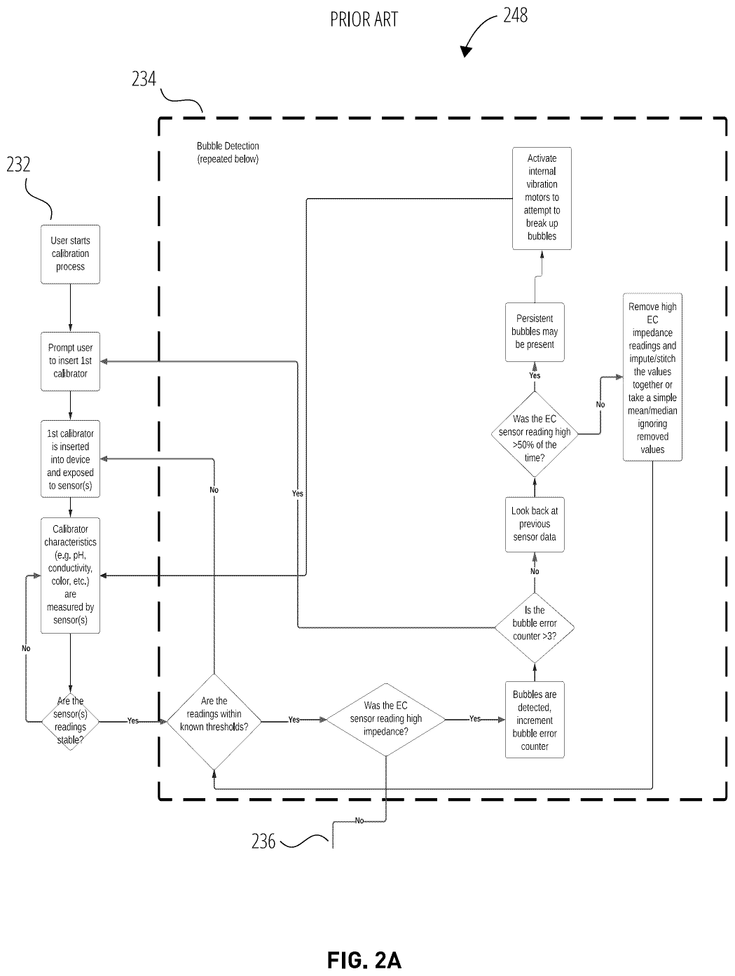

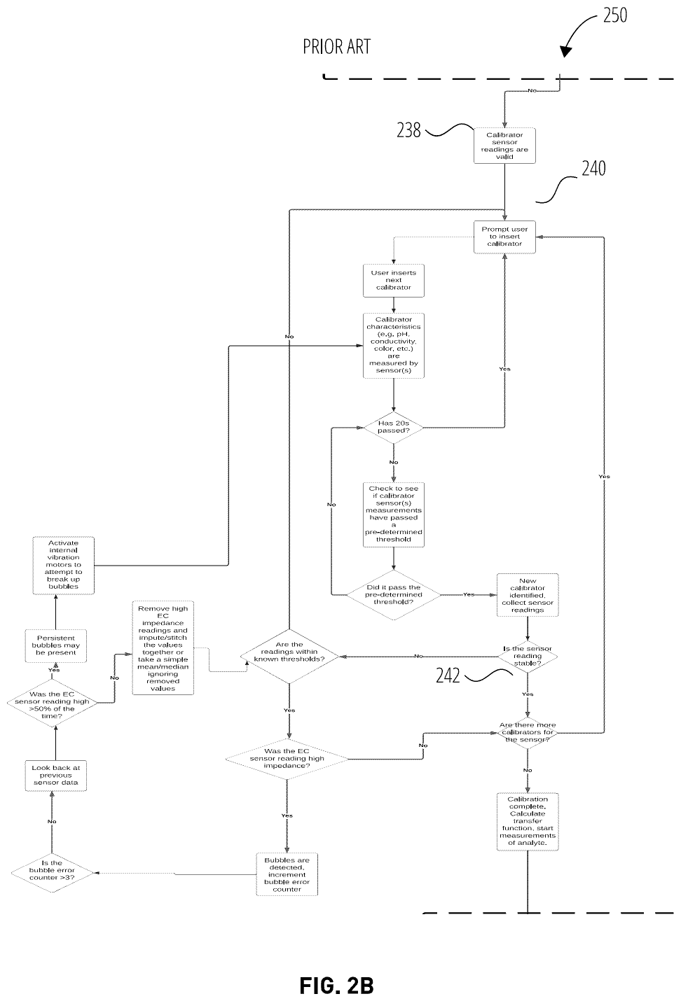

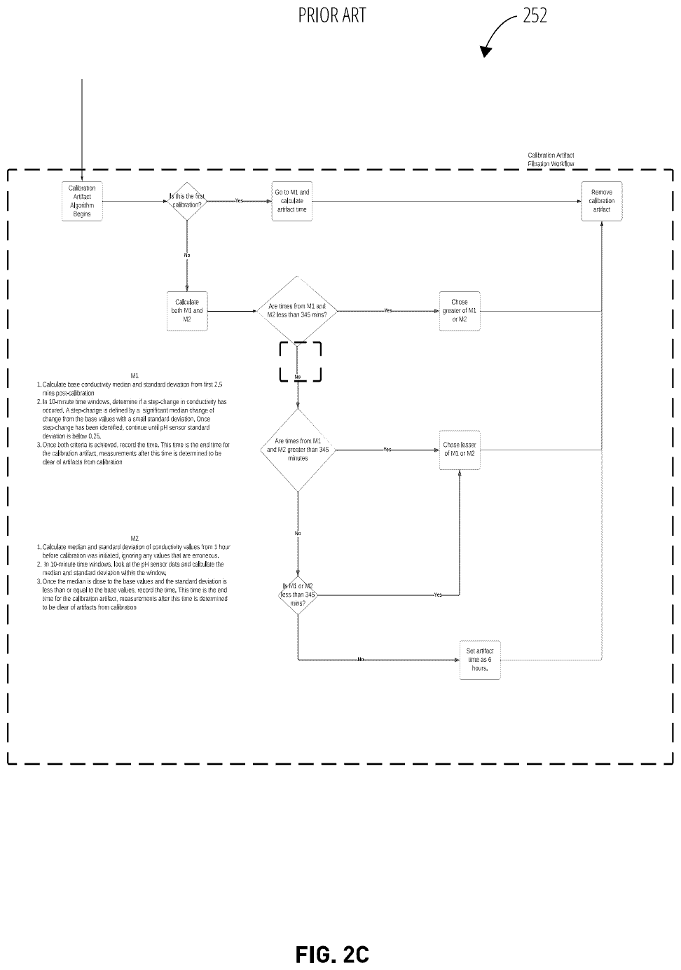

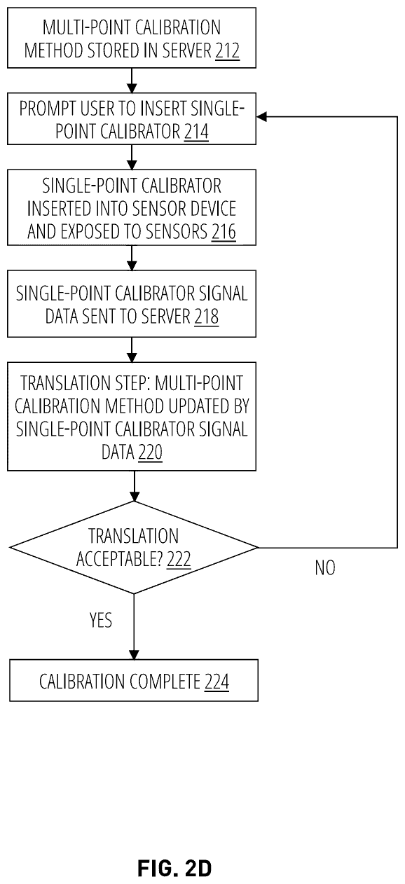

1. Determination of signal stability: Calculate derivative of signal to determine rate of change. Derivative can be calculated instantaneously or after signal smoothing. Choice of whether to apply signal smoothing (and how much) is based on the sensor response time and sampling rate. The faster the sampling rate and the response time, the more smoothing should be applied to reduce the chance of false stability.
2. Determination of contaminants: Contaminants can be detected by outlier detection using traditional statistical techniques or machine learning.
3. Determination of bubbles present: Electrical conductivity sensors (or similar) can be used to detect air bubbles that would cause erroneous calibration measurements. Air bubbles typically present as high impedance measurements with the EC sensor.
4. Temperature correction: Sensors or analytes may be sensitive to temperature. Therefore, there needs to be a process to calibrate in an environment with controlled temperatures or implement an algorithm that corrects for the temperature during calibration.
5. Linearity: For sensors with a linear response, checking linearity after calibration verifies that the correct calibrator values were read. It also helps to ensure accuracy, and can help detect any nonlinearities.
6. Determination of Sensor Status: Based on the response when exposed to calibrators, the sensor condition can be determined by comparing to a known range of historical normal values.
The multi-point calibration as illustrated in FIGS. 2A-C may comprise various steps, to ensure a robust calibration including, but not limited to, data validation 248, bubble detection 234, and artifact detection 252, described below.

FIG. 2A to 2C relate to EC and pH sensor calibration methods, which may be applied generally to any calibration method whether it be multi-point calibration or single-point calibration. It should be readily understood that this method may be applied to calibration of other sensors, such as UV-VIS, optical sensors, lactate, amylase, glucose, and the like, that require external calibrators.

Instructions for performing the method may be stored on a computer readable, non-transitory medium, that, when executed by a computer, causes it to perform the steps described below.

Broadly, the steps of calibration method may be described as two alternating steps, and a third step. Alternating data validation (FIG. 2A) and data calibration (FIG. 2B) steps are performed for each pair of validation and calibration steps corresponding to a calibration or reference fluid flowing through the flow cell and being measured by sensors in a flow cell. Moreover, these alternating steps are performed for each calibration or reference fluid before moving forward with performing the same with each subsequent calibration or reference fluid. The calibration data is then filtered/augmented in a third, filtering/augmentation step (FIG. 2C). For multiple types of calibration fluids, the third step may be performed after all of the data validation and calibration steps.

Generally, the flow charts in FIG. 2 A-C relate to calibration methods that may be applied to a plurality of sensors and calibrators. In the case of in-situ calibration, each sensor is calibrated with one calibrator or calibration fluid.

The method for calibration of a multi-port stopcock flow cell device 108 generally comprises executing, by a computing device, instructions stored on the memory, which cause the processor to perform the steps:

1. Prompt a user to insert a calibration fluid into an input port of a multi-port stopcock flow cell device 108, the calibration fluid flow diverted via a mechanism for diverting flow, through a flow cell comprising one or more fluid channels, the flow cell fluidically connected to the multi-port stopcock flow cell device 108, the multi-port stopcock flow cell device 108 comprising one or more sensors for measuring data relating to the calibration fluid;
2. Receive, via a connection mechanism connecting the one or more sensors to the computing device, the measured data;
3. Determine whether the measured data is valid, and upon the data being valid, repeat steps 1-3 until the user indicates that there are no more calibration fluids;
4. Filter the calibration data based on pre-determined thresholds.

In view of the specific example of conductivity in FIGS. 2A-C, the method involves the following:

FIG. 2A: Data validation 248

1. Execution 232: The method first prompts a user to insert a first calibrant. The user may be prompted with instructions on a display from a digital application in a computing device or an indicator on the device itself. The user may allow calibration fluid to flow through port C, and divert the stopcock such that the calibration fluid flows over the biosensors. The method comprises one or more instances of a user inserting a calibrator into the calibration port of the multi-port stopcock flow cell device 108, the sensors in the flow cell measure data relating to the calibrant (for example, conductivity of a buffer solution) and send the data to the computing device.
2. Bubble detection 234: Ensuring stable readings, determining whether bubbles are detected in the flow cell by abnormal readings, such as high Electrical Conductivity (EC) readings. Abnormal readings may be pre-set thresholds, or may be learned by the method based on the average (or other metric) of other readings. Upon determining that readings are abnormal and the data is invalid, the method may perform corrective actions, such as activating an active bubble removal means.
3. Next step 236: When the readings are stable, within thresholds, and are not abnormal, the calibration data may be sent to the next step (described in FIG. 2B).

FIG. 2B: Data Calibration 250

1. Following the data validation step 212, valid data is saved for processing 238.
2. Calibration prompt 240 prompts a user to insert another calibrator. If there is another calibrator, repeat steps 1-3 of FIG. 2A. Otherwise, proceed to next step.
3. Calibration complete 242 when there are no more calibrants to be added.

FIG. 2C: Calibration Artifact Filtration 252—Following the calibration and data validation steps, the valid data that was saved for processing from step 1 of the data calibration portion of the method, is filtered via the calibration artifact filtration, based on pre-set thresholds, including, but not limited to, one or more of: time, stability, standard deviation, mean, median, and the like.

The thresholds, M1 and M2, are established based on the following steps:
i. M1:
   a. Calculate base conductivity median and standard deviation from a first time threshold past calibration (i.e. 2.5 minutes), or after a number of samples (i.e. after 5 calibration samples have been run).
   b. In a pre-set time window (i.e. 10 minutes), determine if step-change in conductivity (significant median of change from base values, with small standard deviation) occurred. Then continue until pH sensor standard deviation is below a threshold (in this case 0.25).
   c. Once both criteria are achieved, record time—this is the end time for the calibration artifact: measurements after the end time are determined to be clear of artifacts from calibration and will be reflective of the actual analyte being measured.
ii. M2:
   a. Calculate median and standard deviation of conductivity values from a time before initiating calibration (i.e. 1 hour), ignoring erroneous values.
   b. In pre-set time windows (i.e. 10 minutes), calculate median and standard deviation of data within the window.
   c. When the median is close to the base values and the standard deviation is less than or equal to the base values, record the time. This time is the end time for the calibration artifact-measurements after this time are determined to be clear of artifacts from calibration and will be reflective of the actual analyte being measured.

FIG. 2D illustrates a block diagram of a method for a single point, on-site calibration of a sensor device in accordance to one embodiment. A user may perform a final, single-step calibration with a single-point calibrator, which may be used to translate the multi-point calibration to an up-to-date calibration curve. The point of care step may be completed at a location remote from the factory calibration step. In some embodiments, once the results of the multi-point calibration are stored on a server 212, a user is prompted to insert a single-point calibrator 214 into a sensor device, allowing the calibrator to be exposed to one or more sensors within the sensor device 216. Calibration is then initiated, and the resulting single-point calibration data is sent to the server 218. Once stored in the server, a translation step follows wherein the multi-point calibration results are updated by the single-point calibrator signal data 220. Whether translation is acceptable is determined 222 thereafter. If translation is acceptable, single-point calibration is deemed as complete. Otherwise, the method repeats from the point of prompting a user to insert the single-point calibrator 214 into the sensor device until translation is acceptable. Generally, translation is deemed acceptable based on criteria such as specific characteristics of sensors involved and ensuring that the updated calibration parameters fall within values obtained from statistical analysis of a sensor population. For example, one such criterion involves checking if the slope of the pH sensor is within +/−95-105% of 59 mV/pH.

In other embodiments of the single-point, on-site calibration method, the in-situ calibration or single-step calibration with a single point calibrator may be initiated by the user at any point in time without the need to have the results of the multi-point calibration stored on a server first. In some embodiments, the multi-point calibration data and single-point calibration data may be stored directly in the sensor device, while the multi-point calibration data may further be encoded on a barcode such as a QR code in other embodiments.

Replicate measurements of the single reference standard, e.g. calibrator or calibrant, may be required if the single-point method detects a difference between the expected difference of the calibrant and multi-point calibration curve, or artifacts, or other anomalies. However, generally, the user is prompted to insert only one reference sample during on-site calibration.

It is advantageous to minimize calibration by end-users to minimize user burden and error. In order to successfully do this, multi-point calibrations done in the factory need to be translated to a single point calibration that is done by the user.

The in-situ calibration or single point calibration process may comprise one or more of the following steps:

a. Retrieve multi-point calibration data from device/gateway/cloud or from a barcode such as a QR code. The multi-point calibration data needs to be present before proceeding with the next steps. The calibration data is subsequently checked after retrieval to determine whether the values are valid. For example, they are checked to see if the calibration data or information is not corrupted and that the values fall within an expected range of values given a sensor type. The calibration information is then matched with the information of the sensor(s) that are currently used (type, serial number, etc.).
b. Proceed with the single-point calibration process. Each sensor will need a single point calibration. In some embodiments, multiple different calibrators may be combined into 1 so that multiplexed sensors can be calibrated using only 1 calibrator (e.g. pH/EC can be calibrated using a combined pH 7 and 12.88 mS/cm calibration fluid). Other examples of calibrators may include pH buffers (phosphate-based, borate-based, TRIS, etc.), conductivity standard solutions (KCl-based, NaCl-based, etc.) and colorimetric standard solutions.
c. Check for valid calibration. Similar to the factory calibration checks, a stable signal, valid temperature range, and valid signal (i.e. no bubbles, contamination etc.) may be checked. A check to determine if the sensor readings are within expected range will also be done based on historical data or a machine-learning/regression-based prediction based on historical data.
d. Temperature and drift corrections of the raw sensor output. At this step, the stable raw sensor readings should be corrected for temperature and drift effects. This can be done using a look up table or an equation based on historical data or first-principles.
e. Corrections of calibrator values. Nominal value of the calibrator may be corrected based on temperature using a look up table or known equations. The drift in the calibrator can also be corrected at point of use by: comparing to a reserved sample from the same lot and updating the values over-the-air, using a look up table or by applying known correction models.
Each calibrator may be assigned an expiry date, whereby the calibrator can be used within that time period. However, the calibrator itself might be drifting (ever so slightly or otherwise) during that time. To make the measurements even more accurate, each time a calibrator batch is made, some number of samples with the corresponding lot code are saved, and periodically tracked and tested in the lab/factory to obtain current calibrator information, e.g. calibration curve. When the actual calibrator is used in the field to calibrate a device, the actual value of the calibrator can be determined and/or updated by taking the lot code and determining or retrieving the current calibrator information, e.g. calibration curve, from the samples set aside after manufacturing, and using those values as a baseline (in addition to temperature correction, etc.). In some embodiments, the current calibration information may be saved on a server and retrieved at any point of calibration. In other embodiments, the current calibration information may be encoded on barcodes such as QR codes which may be scanned prior to calibrations to retrieve the information.
f. Slope/Offset corrections: Correct the slope or offset of the sensor (the choice would be based on the inherent characteristics of the sensor. e.g. some pH sensors may be relatively insensitive to slope changes, therefore the translation process may correct offset) based on the sensor reading, temperature of the measurement).
g. Saving new model: The new model can be saved on-device/chip, gateway/computing device and/or stored to the cloud.

If subsequent calibrations are needed, as determined by a “Translation acceptable?” step 222 the user can be prompted to repeat the process.

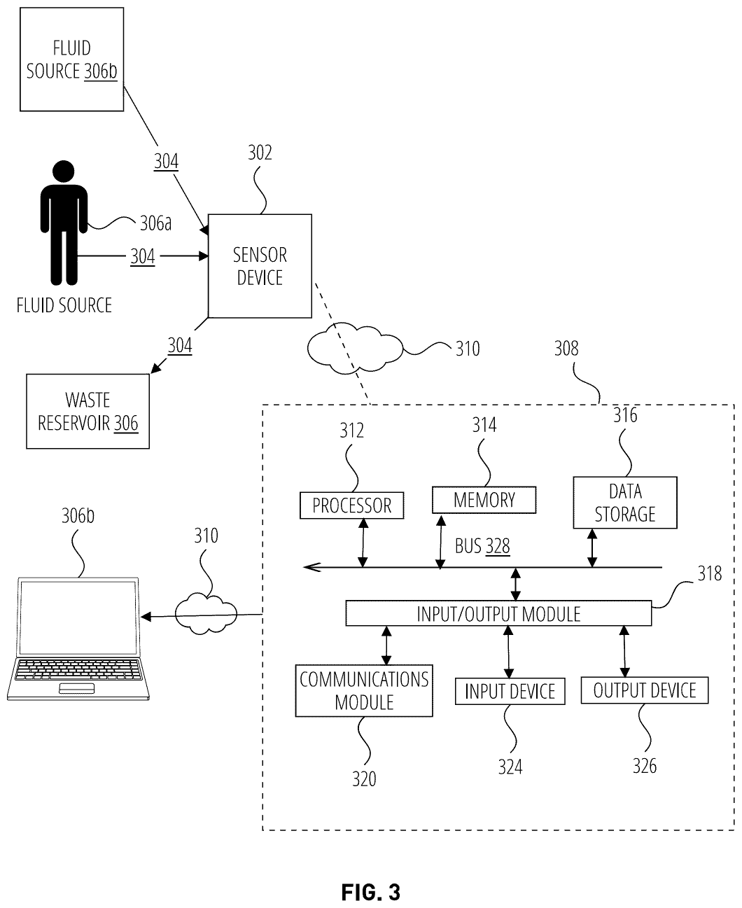

FIG. 3 is a schematic diagram illustrating a system 312 with which aspects of the disclosure may be implemented. According to an embodiment, fluid 304 flows from a fluid source 308 a and 308b (for example, a patient's biofluid 308a if in data acquisition mode, or from a calibration fluid source 308b if in calibration mode, or a cleaning fluid source 308b if in cleaning mode), to a sensor device 302, where it flows over one or more sensor elements in the sensor device 302. Fluid 304 from the fluid source (308a, 308b) flows through the one or more sensor elements in the sensor device 302 through a fluid channel and exits to a waste reservoir 306. Data pertaining to bioanalytes in the fluid is measured by the sensor elements and sent to a processor 316 of the computer system 312, where the patient data is processed, or the calibration process is performed.

The computer system 312 may be implemented using hardware or a combination of software and hardware, either in a dedicated server, integrated into another entity, or distributed across multiple entities. The computer system 312, or aspects of it, may be integral to the sensor device 302, or separate from it. The sensor device 302 may communicate with the computer system 312 via a communication mechanism 314 wirelessly over a network, or via a wired communication mechanism.

Computer system 312 (e.g., server and/or client) includes a bus 330 or other communication mechanism for communicating information, and a processor 316 coupled with bus 330 for processing information. By way of example, the computer system 312 may be implemented with one or more processors 316. Processor 316 may reside in the sensor device 302, in the computer system 312, or both. Data may be pre-processed in the sensor device 302, for example by correction of data based on on-site conditions, and then sent to a computer system 312 for more rigorous processing. Processor 316 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 312 can include, in addition to hardware, code that creates an execution environment for the computer program in question, e.g., code that constitutes processor firmware, a protocol stack, a database management system, an operating system, or a combination of one or more of them stored in an included memory 318, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus 330 for storing information and instructions to be executed by processor 316. The processor 316 and the memory 318 can be supplemented by, or incorporated in, special purpose logic circuitry. The sensor device 302 may have its own memory 318 for storing data. The memory 318 may be in a separate computer system 312. The memory 318 may comprise cloud data storage 320.

The instructions may be stored in the memory 318 and implemented in one or more computer program products, i.e., one or more modules of computer program instructions encoded on a computer readable medium for execution by, or to control the operation of, the computer system 312, and according to any method well-known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory 318 may also be used for storing temporary variable or other intermediate information during execution of instructions to be executed by processor 316.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 312 may further includes data storage 320 such as a magnetic disk or optical disk, coupled to bus 330 for storing information and instructions. Computer system 312 may be coupled via input/output module 322 to various devices, including a display element 310, such as a phone screen or computer screen. The input/output module 322 can be any input/output module. Exemplary input/output modules 322 include data ports such a USB ports. The input/output module 322 is configured to connect to a communications module 324. Exemplary communications modules 324 include networking interface cards, such as Ethernet cards and modems. In certain aspects, the input/output module 322 is configured to connect to a plurality of devices, such as an input device 326 and/or an output device 328. Exemplary input devices 326 include a keyboard and a pointing device, e.g., a mouse or a trackball, by which a user can provide input to the computer system 312, Other kinds of input devices 326 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback, and input from the user can be received in any form, including acoustic, speech, tactile, or brain wave input. Exemplary output devices 328 include display devices such as an LCD (liquid crystal display) monitor, for displaying information to the user.

According to one aspect of the present disclosure, the methods for processing analyte data and/or calibrating a system to process analyte data, detected by a sensor device 302, described herein, can be implemented using a computer system 312 in response to processor 316 executing one or more sequences of one or more instructions contained in memory 318. Such instructions may be read into memory 318 from another machine-readable medium, such as a data storage device 320. Execution of the sequences of instructions contained in the main memory 318 causes processor 316 to perform the process steps described herein. One or more processors in a multi-processing arrangement may also be employed to execute the sequences of instructions contained in memory 318. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., such as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. The communication network can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system 312 can include clients and servers. A client and server are generally remote from each other and typically interact through a communication network. The relationship of client and server arises by virtue of computer programs running on the respective computers and having a client-server relationship to each other. Computer system 312 can be, for example, and without limitation, a desktop computer, laptop computer, or tablet computer. Computer system 312 can also be embedded in another device, including the sensor device 302, for example.

The term “machine-readable storage medium” or “computer readable medium” as used herein refers to any medium or media that participates in providing instructions to processor 316 for execution. Such a medium may take many forms, including, but not limited to, non-volatile media, volatile media, and transmission media. Non-volatile media include, for example, optical or magnetic disks, such as data storage device 320. Volatile media include dynamic memory, such as memory 318. Transmission media include coaxial cables, copper wire, and fiber optics, including the wires that comprise bus 330. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter effecting a machine-readable propagated signal, or a combination of one or more of them.

As the processor 316 reads and processes sensor data (whether from monitoring patient fluid, calibration fluid, cleaning fluid, or a combination thereof), information may be read from the sensor data and stored in a memory device, such as the memory 318. Additionally, data from the memory 318 may be accessed at a customer server, via a network, such as the bus 330, in order to view bioanalyte concentrations and/or any risk assessments determined by the processor 316. Further, data storage 320 may be read and loaded into the memory 318. Although data is described as being found in the memory 318, it will be understood that data does not have to be stored in the memory 318 and may be stored in other memory accessible to the processor 316 or distributed among several media, such as the data storage 320, and may be received at a remote or local server for data processing.

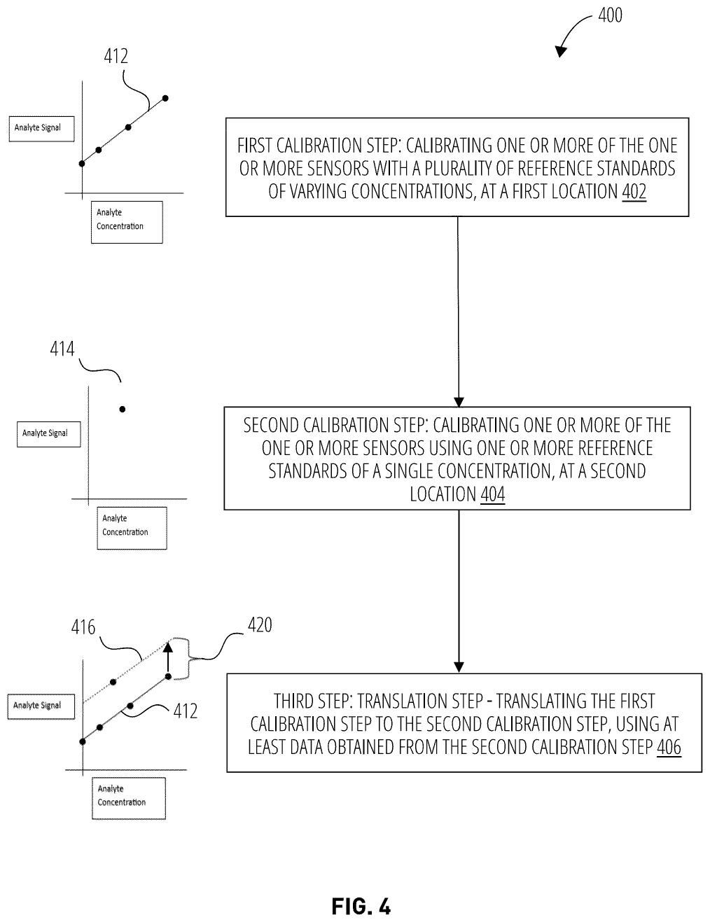

FIG. 4 illustrates a routine 400 for calibrating one or more sensors in accordance with one embodiment. In block 402, routine 400 runs a first calibration step comprises calibrating one or more of the one or more sensors with a plurality of reference standards, e.g. calibrator or calibrants, of varying concentrations, at a first location. Block 402, the first calibration step, yields multi-point calibration curve 412 for each analyte. In block 404, routine 400 runs a second calibration step comprises calibrating one or more of the one or more sensors using one or more reference standards, e.g. calibrator or calibrants of a single concentration, at a second location. Block 404, the second calibration step, yields a single-point calibration 414 for each analyte. In block 406, routine 400 runs a third step comprises translating the multi-point calibration curve to a single-point calibration, using at least data obtained from the second calibration step. Based on the translation 420 resulting from a translation algorithm in block 406, the multi-point calibration curve 412 is translated to a new, translated calibration curve 416, which is the curve used to analyze unknown data (e.g. analytes), on-site.

Methods disclosed herein may employ various decision-making engines which evaluate the calibration data in order to decide which data may be best suited for determining calibration artifacts. For example, very high or very low pH or conductivity may be filtered out of the calibration, and data determined to be invalid due to the presence of bubbles may be filtered out.

Calibrated data from the method disclosed above is preferably used to process fluid data, i.e. from a patient.

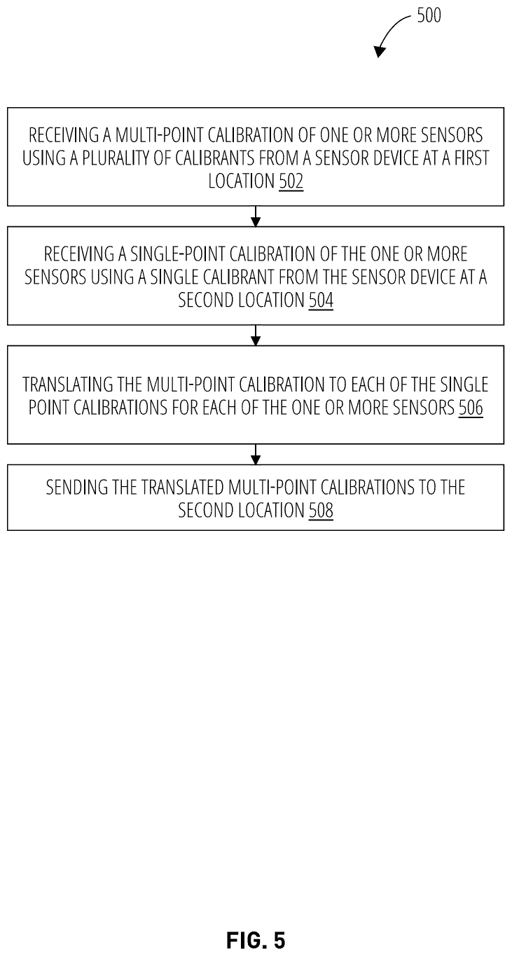

FIG. 5 illustrates a routine 500 for calibrating one or more sensors in accordance with one embodiment. In block 502, routine 500 receives, at a server, the server communicatively coupled to a memory, a multi-point calibration of the one or more sensors using a plurality of calibrants, from a sensor device at a first location. In block 504, routine 500 receives, at the server, single-point calibration of the one or more sensors using a single calibrant for each of the one or more sensors, from the sensor device at a second location. In block 506, routine 500 translates, via a processing unit communicatively coupled with the memory, the multi-point calibration to each of the single-point calibrations for each of the one or more sensors. In block 508, routine 500 sends, via the server, the translated multi-point calibrations to the second location.

Routine 500 may comprise one or more of a software-based algorithm, stored on non-transitory memory and executable by a processor, where sensor drift/changes are corrected for based on initial factory calibration, imputing processes wherein a stable signal in any calibration process can be imputed if there is a bubble that causes an anomaly within the reading.

Similarly, the final stable output of a sensor during calibration can be estimated by measuring for a nominally short time frame, then estimating where it will settle by using a known model (e.g. extrapolation based on known sensor response characteristics) from first-principles or empirical experimentation. This will shorten the overall time it takes for calibration to be complete.

Any of the calibration and correction processes and methods disclosed herein can be done on-chip/sensor device, via a network gateway, a cloud platform or similar.

The multi-point calibration data may be saved to the sensor device, specifically on a memory of the sensor device. Alternatively, the multi-point calibration data may be saved on a gateway (such as an app, or the cloud, communicatively coupled to the sensor device via a network or a wired connection mechanism), such that the sensor device may access it during the single-point calibration step.

Generally, the multi-point calibration method comprises a pre-calibration step wherein it is determined, based on, for example, the number or kinds of sensors present in the sensor device, one or more of the following multi-point calibration method parameters: the calibrators that are needed (and the interactions between them), delivery or type of flow which may be single continuous or intermittent (i.e. interrupted with air or some other flushing agent), the checks for the correctness of sensor data and/or calibrator values that are needed.

In some embodiments, the multi-point calibration method is configured, by one or more algorithms, based on one or more inputs, to automatically determine the multi-point calibration method parameters.

In an embodiment, the calibration method is stored on a computer system, which is coupled to the sensor device, and receives the one or more inputs via communication with the sensor device. In this embodiment, reading the sensor device inputs and determining the multipoint calibration method parameters may be automatic.

In another embodiment, the multi-point calibration method may comprise an additional step of prompting a user (such as a lab technician), to flow specific calibrants (also referred to as calibrators herein) or rinsing (also referred to as cleaning fluids herein) through the sensor device at either set times, or times determined by the algorithm based on measured sensor data.

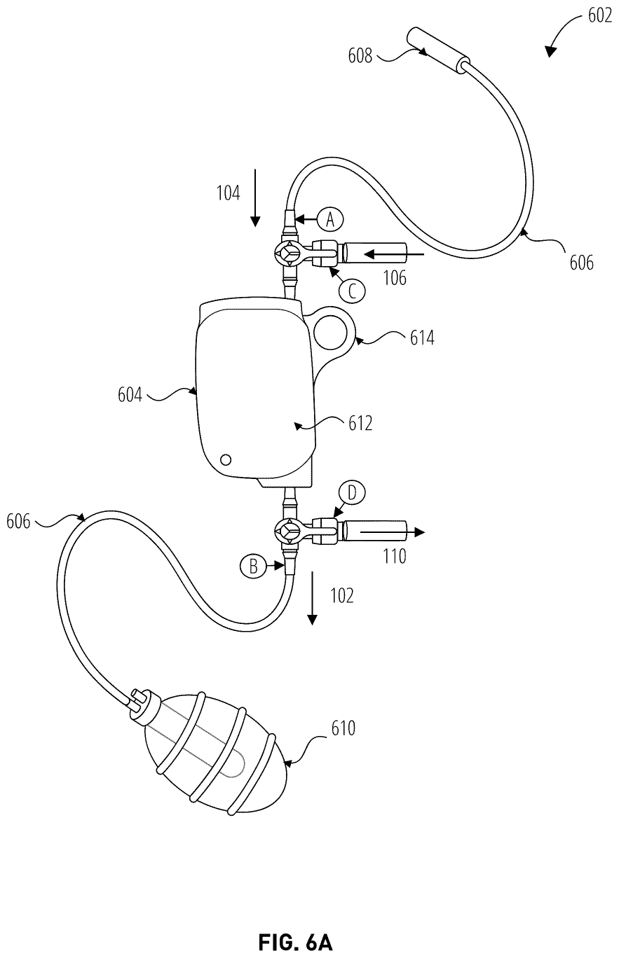

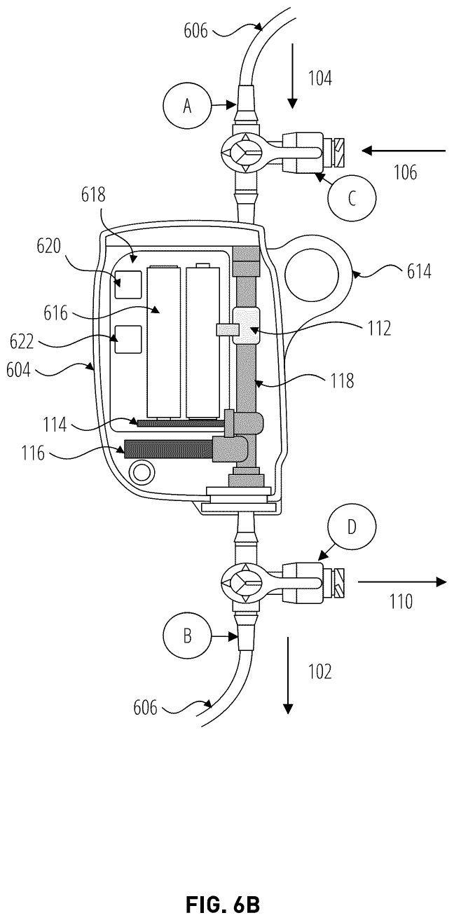

FIG. 6A illustrates a system 602 including an inline sensor device 604 in accordance with one embodiment. The system 602 further comprises catheter 606, wound drain 608 and reservoir 610. The system 602 further incorporates the use of a different configuration of the multi-port stopcock flow cell device 108 shown in FIG. 1. The different configuration is based on the placement of one or more sensors relative to the inlet and outlet ports which is more evidently shown in FIG. 6B. Although the configuration of the multi-port stopcock device is different from that shown in FIG. 1, it comprises the same components such as one or more inlet port (A, C) and one or more outlet ports (B, D) and a mechanism (i.e. a lever) for diverting flow of fluid between ports through the fluid channel are shown as part of the inline sensor device 604.

System 602 may be used to determine a patient's condition, such as a clinical condition. Such condition may be an occurrence of a post-operative leak based at least in part on data from one or more sensors within the inline sensor device 604. The one or more sensors (shown in FIG. 5B) may be placed within the catheter 606 which allow the patient's bodily fluids to be monitored. The inline sensor device 604 may comprise one or more inlet ports (A, C), one or more outlet ports (B, D), a hook 614, an outer shell 612 housing internal components comprising at least one or more sensors. In the embodiment shown in FIG. 6A, the patient's bodily fluids flow into 104 the inline sensor device 604 through inlet port A, and flow out 102 the inline sensor device 604 through the outlet port B. Furthermore, calibrants or cleaning fluid may flow into 106 the inline sensor device 604 through inlet port C and flow out 110 the inline sensor device 604 through output port D. By means of a lever located at the junction between the one or more inlet ports and one or more outlet ports, the flow of fluid may be controlled by a user. In some embodiments, the reservoir 610 may be, for example, a bulb, balloon or drainage bag. Fluids can be drained using with or without negative pressure being applied to the system.

FIG. 6B illustrates a magnified view of internal components within the inline sensor device shown in FIG. 6A. Compared to the multi-port stopcock flow cell device 108 in FIG. 1, that integrated within the inline sensor device 604 comprises one or more sensors (biosensor e.g. pH electrode 116, biosensor e.g. pH reference electrode 114, and biosensor e.g. electrical conductivity 112) that are placed differently relative to the one or more inlet ports (A, C) and one or more outlet ports (B, D). In the embodiment shown in FIG. 6B, the biosensor 112 is placed more proximate to the one or more inlet ports (A, C), while biosensor 114 and biosensor 116 are more proximate to the one or more outlet ports (B, D). In this embodiment, the one or more sensors 112, 114, 116 are capable of measuring fluid properties, biomarkers or other analytes. The one or more inlet ports comprises a patient fluid inlet port (A), a patient fluid outlet port (B), a calibration/cleaning inlet port (C) and a calibration/cleaning outlet port (D). The one or more inlet ports (A, C) generally receives fluid from a fluid source, such as a patient, wherein the one or more inlet ports (A, C) are fluidically connected by a flow cell 118 to the one or more outlet ports (B, D). There is also a mechanism (i.e. a lever) for diverting flow of fluid between ports through the flow cell 118 so that the one or more sensors can measure different analytes from a fluid or be calibrated by one or more calibrators. The flow cell 118 houses sensors 112, 114, 116. In some embodiments, the inline sensor device 604 may comprise one or more batteries 616 to power the inline sensor device 604 and a printed circuit board 618 comprising at least one or more processors 620, a memory 622 and a connection mechanism to the sensors 112, 114, 116.

In some embodiments, upon fluid (patient fluid or calibration fluid) flowing through the flow cell 118 and over the sensors 112, 114, 116, the sensors measure data relating to the fluid and send the measured data, via the connection mechanism, to a server through a network or a memory on the inline sensor device 604, wherein the server and the memory are coupled to one or more processors. In some embodiments, the calibration method used to calibrate the one or more sensors 112, 114, 116 in the inline sensor device 604 is based on the method described in FIG. 2D.

In some embodiments, the inline sensor device 604 may be an inline modular device, wherein the inline modular device comprises one or more swappable sensor modules and a processing module. The one or more swappable sensor modules would enclose the one or more sensors and the multi-port stopcock flow cell assembly, while the processing module would enclose components such as a power source, a memory and one or more processors that may be part of a printed circuit board.

Machine learning algorithms may be applied to previously acquired signal data associated with a user condition or calibration anomalies. For example, pattern recognition may be performed on previously acquired signal data that is associated with a particular sensor condition. The machine learning algorithms may generate a user condition classification model trained by the previously acquired signal data. The machine learning algorithms may generate a calibration anomaly model trained by previously acquired calibration data. The machine learning algorithms may determine pre-calibration processes and calibration protocols, trained by previously acquired calibration data.

Any of the models, processes, or protocols, may be displayed on a display element 310 (such as a computer or tablet screen) to prompt a user to load a certain calibrant/reference material/cleaning fluid, or to prompt the user to open fluid flow from the patient to the sensor device when cleaning is complete.

These algorithms may include, for example, deep learning architectures such as Deep Belief Network (DBN), Stacked Auto Encoder (SAE), Convolutional Neural Network (CNN) or Recurrent Neural Network (RNN) may be used. Other examples include, without limitation, Restricted Boltzmann machines (RBM), Social Restricted Boltzmann Machines (SRBM), Fuzzy Restricted Boltzmann Machines (FRBM), TTRBM models of Deep Belief Networks (DBN) or similar approaches could be used; AE, FAE, GAE, DAE, BAE models of Statistically Adjusted End Use (SAE) models could be used; models such as AlexNet, ResNet, Inception, VGG16, ECNN models of CNN may be used; Bidirectional Recurrent Neural Networks (BiRNN), Long Short-Term Memory (LSTM) networks, Gate Recurrent Unit (GRU) of RNN may also be used. Additional techniques specific to time-series modelling may be employed, including, but not limited to, dynamic time warping, change point detection, Autoregressive Integrated Moving Average (ARIMA).

In some embodiments, other types of algorithms such as physics-based mathematical computations and basic multiple linear regression models may also be relied upon in conjunction with or in complementarity with those architectures and learning algorithms. This may further include cumulative average (CA) methods.

The present disclosure includes systems having processors to provide various functionality to process information, and to determine results based on inputs. Generally, the processing may be achieved with a combination of hardware and software elements. The hardware aspects may include combinations of operatively coupled hardware components including microprocessors, logical circuitry, communication/networking ports, digital filters, memory, or logical circuitry. The processors may be adapted to perform operations specified by a computer-executable code, which may be stored on a non-transitory computer readable medium.

The steps of the methods described herein may be achieved via an appropriate programmable processing device or an on-board field programmable gate array (FPGA) or digital signal processor (DSP), that executes software, or stored instructions. In general, physical processors and/or machines employed by embodiments of the present disclosure for any processing or evaluation may include one or more networked or non-networked general purpose computer systems, microprocessors, field programmable gate arrays (FPGA's), digital signal processors (DSP's), micro-controllers, and the like, programmed according to the teachings of the exemplary embodiments discussed above and appreciated by those skilled in the computer and software arts. Appropriate software can be readily prepared by programmers of ordinary skill based on the teachings of the exemplary embodiments, as is appreciated by those skilled in the software arts. In addition, the devices and subsystems of the exemplary embodiments can be implemented by the preparation of application-specific integrated circuits, as is appreciated by those skilled in the electrical arts. Thus, the exemplary embodiments are not limited to any specific combination of hardware circuitry and/or software.

Stored on any one or a combination of computer readable media or non-transitory computer readable media, the exemplary embodiments of the present disclosure may include software for controlling the devices and subsystems of the exemplary embodiments, for processing data and signals, for enabling the devices and subsystems of the exemplary embodiments to interact with a human user or the like. Such software can include, but is not limited to, device drivers, firmware, operating systems, development tools, applications software, and the like. Such computer-readable media further can include the computer program product of an embodiment of the present disclosure for preforming all or a portion (if processing is distributed) of the processing performed in implementations. Computer code devices of the exemplary embodiments of the present disclosure can include any suitable interpretable or executable code mechanism, including but not limited to scripts, interpretable programs, dynamic link libraries (DLLs), complete executable programs and the like.

Common forms of computer-readable media may include, for example, magnetic disks, flash memory, RAM, a PROM, an EPROM, a FLASH-EPROM, or any other suitable memory chip or medium from which a computer or processor can read.

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Information as herein shown and described in detail is fully capable of attaining the above-described object of the present disclosure, the presently preferred embodiment of the present disclosure, and is, thus, representative of the subject matter which is broadly contemplated by the present disclosure.

What is claimed is:

1. A method for calibrating one or more sensors in a sensor device fluidically in-line with a patient, the method comprising:
calibrating, at a first location, each of the one or more sensors with a plurality of variable concentration reference calibrators to generate multi-point calibration data;
calibrating, at a second location, each of the one or more sensors using a single-concentration reference calibrator to generate single-point calibration data;
translating, by at least one processor communicatively coupled to a memory, the multi-point calibration data and the single-point calibration data to translated calibration data; and
employing, at the second location, the translated calibration data to analyze one or more analytes detected by one or more calibrated sensors in the sensor device.

2. The method of claim 1, wherein each calibrating step comprises validating calibration accuracy of the one or more sensors.

3. The method of claim 1, wherein the translating step further comprises performing one or more of: slope corrections, offset corrections, temperature corrections, and drift corrections.

4. The method of claim 1, further comprising determining, prior to calibrating each of the one or more sensors, types of the one or more sensors, the plurality of variable concentration reference calibrators, and the one or more single concentration reference calibrators.

5. The method of claim 1, further comprising determining, prior to calibrating each of the one or more sensors, a sequence in which each of the reference calibrators from the plurality of variable concentration reference calibrators are applied to the one or more sensors, wherein the sequence is based on cross sensitivities of the one or more sensors with each of the reference calibrators from the plurality of variable concentration reference calibrators.

6. The method of claim 1, further comprising determining, prior to calibrating each of the one or more sensors, a type of flow in which the plurality of variable concentration reference calibrators is applied to the one or more sensors, wherein the type of flow is a continuous flow or an intermittent flow.

7. The method of claim 1, wherein the plurality of variable concentration reference calibrators is periodically tested to obtain current calibration values, wherein the current calibration values are retrieved prior to generating the multi-point calibration data.

8. A computer-implemented method for calibrating one or more sensors in a sensor device fluidically in-line with a patient, the method comprising:
storing, in a memory coupled to at least one processor, multi-point calibration data generated from calibrating, at a first location, each of the one or more sensors with a plurality of variable concentration reference calibrators;
storing, in the memory coupled to the at least one processor, single-point calibration data generated from calibrating, at a second location, each of the one or more sensors using a single-concentration reference calibrator;
determining, by the at least one processor, translated calibration data based on the multi-point calibration data and the single-point calibration data; and
analyzing, by the at least one processor, one or more analytes detected by one or more calibrated sensors in the sensor device based on the translated calibration data.

9. The computer-implemented method in claim 8, wherein each calibrating step comprises validating calibration accuracy of the one or more sensors.

10. The computer-implemented method in claim 8, wherein the determining translated calibration data further comprises performing one or more of: slope corrections, offset corrections, temperature corrections, and drift corrections.

11. The computer-implemented method in claim 8, further comprising receiving the multi-point calibration data and the single-point calibration data from a data source, wherein the data source is one of: a server through a network, the sensor device or a barcode.

12. The computer-implemented method of claim 8, further comprising receiving, from the data source, pre-calibration information generated from pre-calibration steps comprising:
determining types of the one or more sensors, the plurality of variable concentration reference calibrators, and the one or more single concentration reference calibrators;
determining a sequence in which each of the reference calibrators from the plurality of variable concentration reference calibrators are applied to the one or more sensors; and
determining a type of flow in which the plurality of variable concentration reference calibrators is applied to the one or more sensors.

13. A system for calibrating one or more sensors, the system comprising:
a sensor device fluidically in-line with a patient comprising the one or more sensors for detecting one or more analytes;
a memory; and
at least one processor coupled to the memory comprising program instructions, wherein the program instructions are executable by the at least one processor to perform operations comprising:
storing multi-point calibration data generated from calibrating, at a first location, each of the one or more sensors with a plurality of variable concentration reference calibrators;
storing single-point calibration data generated from calibrating, at a second location, each of the one or more sensors using a single-concentration reference calibrator;
retrieving the multi-point calibration data and the single-point calibration data from the memory;
determining translated calibration data based on the multi-point calibration data and the single-point calibration data; and
analyzing the one or more analytes detected by the one or more calibrated sensors in the sensor device based on the translated calibration data.

14. The system of claim 13, wherein each calibrating step comprises validating calibration accuracy of the one or more sensors.

15. The system of claim 13, wherein the translating step further comprises performing one or more of: slope corrections, offset corrections, temperature corrections, and drift corrections.

16. The system of claim 13, further comprising a data source, wherein the data source is one of: a server through a network, the sensor device or a barcode.

17. The system of claim 13, wherein the operations further comprise determining, prior to calibrating each of the one or more sensors, types of the one or more sensors, the plurality of variable concentration reference calibrators, and the one or more single concentration reference calibrators.

18. The system of claim 13, wherein the operations further comprise determining, prior to calibrating each of the one or more sensors, a sequence in which each of the reference calibrators from the plurality of variable concentration reference calibrators are applied to the one or more sensors, wherein the sequence is based on cross sensitivities of the one or more sensors with each of the reference calibrators from the plurality of variable concentration reference calibrators.

19. The system of claim 13, wherein the operations further comprise determining, prior to calibrating each of the one or more sensors, a type of flow in which the plurality of variable concentration reference calibrators is applied to the one or more sensors, wherein the type of flow is a continuous flow or an intermittent flow.

20. The system of claim 13, wherein the plurality of variable concentration reference calibrators is periodically tested to obtain current calibration values, wherein the current calibration values are retrieved prior to generating the multi-point calibration data.
